(19) United States
(12) Patent Application Publication
(10) Pub. No.: US 2014/0303692 A1
(43) Pub. Date: Oct. 9, 2014

(54) Therapeutic Laser Treatment Device

(71) Applicant: Biolase, Inc., Irvine, CA (US)

(72) Inventors: Federico Pignatelli, Beverly Hills, CA (US); William E. Brown, JR., Roswell, GA (US)

(21) Appl. No.: 14/245,545

(22) Filed: Apr. 4, 2014

Related U.S. Application Data
(60) Provisional application No. 61/808,886, filed on Apr. 5, 2013.

(51) Int. Cl. A61N 5/06

(52) U.S. Cl. CPC A61N 5/0613; A61N 2005/067
USPC 607/89; 607/90

(57) ABSTRACT

A therapeutic device includes a garment configured to be worn on a user's body over a treatment area. The therapeutic device includes a plurality of therapeutic electromagnetic (EM) energy emitting devices, fixed to the garment at locations within the garment for irradiating the treatment area with EM energy when the garment is worn over the treatment area. A power supply is fixed to and within the garment. The power supply stores electrical charge and outputs electrical supply current from the stored electrical charge. A flexible graphite material is located within the garment and conducts the electrical supply current to the EM energy emitting devices to activate the energy emitting devices.

### CROSS-REFERENCE TO RELATED APPLICATION

This application claims priority to U.S. Provisional Patent No. 61/808886, filed Apr. 5, 2013, hereby incorporated herein by reference.

### BACKGROUND

The use of laser therapy for treating certain medical conditions is gaining widespread acceptance within the medical community. Currently, both low level laser therapy (i.e., cold laser therapy) and high level laser therapy are used, for example, to provide temporary pain relief and to accelerate healing. Low level laser therapy typically involves treating the affected area with a low energy (class III) laser that induces little, if any, temperature elevation. In contrast, high level laser therapy typically utilizes a more powerful (class IV) therapeutic laser that is capable of providing deeper penetration and heating of the treatment area. An example of a high level laser that has proven effective for therapeutic treatment is the Biolase™ 10 Pain Therapy Laser sold by Biolase, Inc. of Irvine, Calif.

### SUMMARY

An example therapeutic device includes a garment configured to be worn on a user's body over a treatment area. The therapeutic device includes a plurality of therapeutic electromagnetic (EM) energy emitting devices, fixed to the garment at locations within the garment for irradiating the treatment area with EM energy when the garment is worn over the treatment area. A power supply is fixed to and within the garment. The power supply stores electrical charge and outputs electrical supply current from the stored electrical charge. A flexible graphite material is located within the garment and conducts the electrical supply current to the EM energy emitting devices to activate the EM energy emitting devices.

The flexible conductive material may alternatively comprise at least one of carbon impregnated rubber, carbon impregnated polymer, gold nanoparticles embedded in polyurethane, semiconducting nanowires, a non-woven, an electrically conductive fabric, carbon nanotube-coated silicone, carbon nanotube film, graphene sheets, and metal-nanowire mesh.

In some examples, the emitting devices are high level class IV lasers, or low level class III lasers, or light emitting diodes (LEDs). The garment may be a shoe insole. A controller may control the emitting devices to be activated in a sequence. The sequence may be in a longitudinal direction of the insole or radial direction of the insole. The sequence may yield a ring of light that travels radially outward from a center point or radially inward toward a center point. The power supply may include a piezoelectric device that is located in the insole and that generates, from compression and bending of the insole, electrical charge current for charging the power supply. A controller may direct the electrical supply current, from the power supply, to power the piezoelectric device to vibrate when the EM energy emitting devices are activated. The power supply may include a movement transducer that is located in the insole and that generates, from oscillatory movement of the insole, electrical charge current for charging the power supply. A temperature sensor in the insole may sense temperature, and a controller in the insole, may control activation of the EM emitting devices based on the sensed temperature. A compression sensor in the insole may sense compression force applied by a foot to the insole, and the controller in the insole may control activation of the EM emitting devices based on the sensed compression and count a number of steps taken with the shoe insole based on the sensed compression. The insole may be located in a shoe, and an external controller, outside the shoe, enables a user of the external controller to select treatment control parameters to be communicated to the internal controller, in the shoe, through a wireless communication link between the internal controller and the external controller. The garment might be a glove insert, a bandage, a garment worn on a shoulder, or a garment worn on a lower back.

### BRIEF DESCRIPTION OF THE DRAWINGS

FIG. 1 is a schematic view of a first example electromagnetic (EM) foot treatment device for applying therapeutic EM energy to a foot, the treatment device including a shoe insole.

FIG. 2 is a perspective view of the first EM foot treatment device.

FIG. 3 is a schematic view of a mechanism for electrically charging the first EM foot treatment device from an external power source.

FIG. 4 is a sectional view of a mechanism for electrically charging the first EM foot treatment device from compression force applied to the first EM foot treatment device.

FIG. 5 is a schematic view of a mechanism for electrically charging the first EM foot treatment device from oscillatory movement the first EM foot treatment device.

FIG. 6 is an exploded view of a second example EM foot treatment device.

FIG. 7 is a sectional view of the second EM foot treatment device.

FIG. 8 is an EM treatment device in which therapeutic lasers located in a glove for treating a hand.

FIG. 9 is an EM treatment device in which therapeutic lasers are located in a bandage for treating a body part about which the bandage is wrapped.

FIG. 10 is an EM treatment device in which therapeutic lasers located in a compression sleeve worn on a shoulder for treating the shoulder.

FIG. 11 is an EM treatment device in which therapeutic lasers are located in a garment about a user's waist.

### DETAILED DESCRIPTION

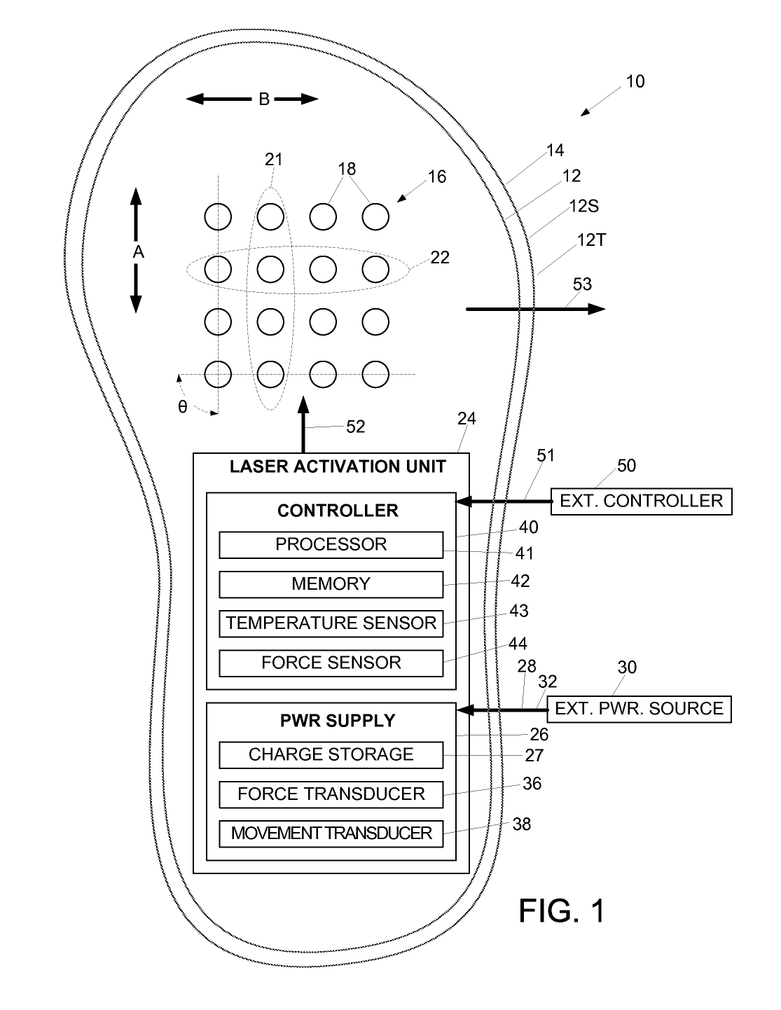

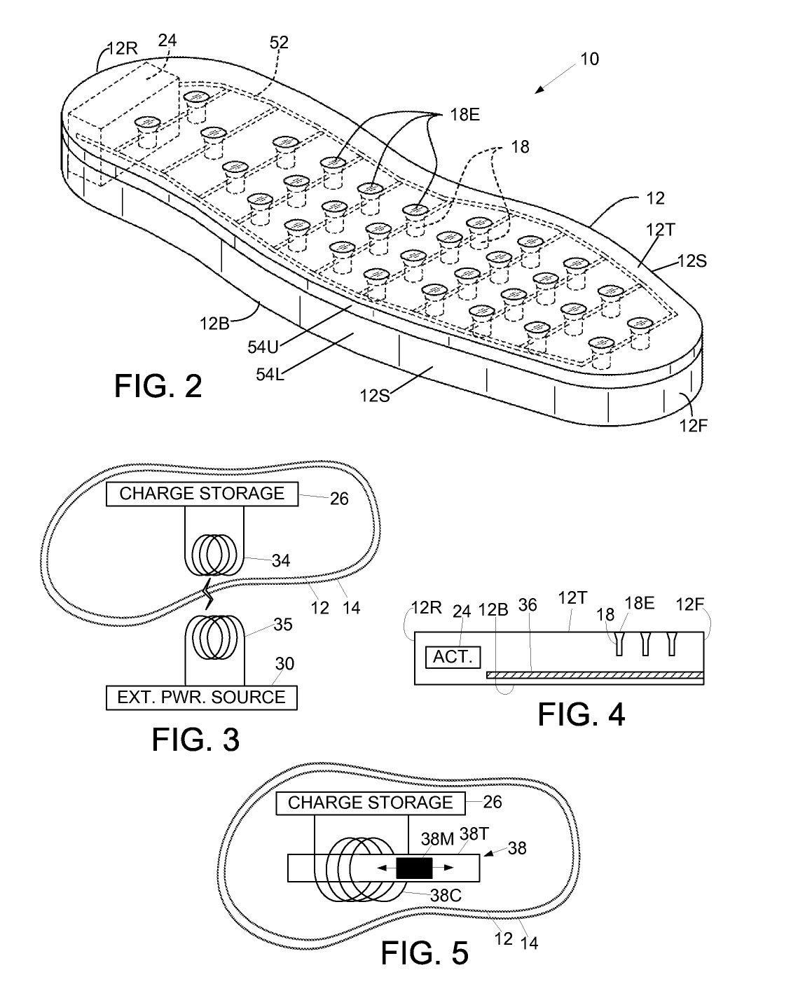

FIGS. 1-11 illustrate different examples of electromagnetic (EM) treatment devices for applying therapeutic EM energy to treatment areas of the body. The EM treatment may provide biostimulation and/or a reduction of pain, bacteria, or onychomycosis. The first example device is a foot treatment device 10, schematically in FIG. 1 and in perspective view in FIG. 2. The foot treatment device includes a footwear item that includes EM emitting devices that apply EM energy to a foot of a user (patient). In this example, the footwear is a shoe insole, the emitting devices are lasers, and the energy is laser energy. A flexible graphite material conducts electrical power from a power source in the insole to the lasers.

As shown in FIGS. 1-2, the example foot EM treatment device 10 includes a shoe insole 12, described with reference to a longitudinal axis “A” and a lateral axis “B”. The insole has top surface 12T, a bottom surface 12B, longitudinally opposite front and rear ends 12F, 12R, and laterally opposite sides 12S. The insole 12 is an orthotic (shoe insert) configured to be inserted by a user into a footwear item, such as a shoe device (e.g., shoe or sandal) or sock. In this example, the footwear item is a shoe with a side wall 14. The insole 12 may alternatively be manufactured as part of footwear item.

An array (plurality) 16 of therapeutic EM energy emitting devices 18 are attached to and embedded in the insole 12, for emitting (applying, irradiating) EM energy (light) to target tissue in the treatment area. The EM energy emitting devices 18 in this example are therapeutic lasers for emitting laser light, specifically high level (class IV) therapeutic lasers. Other EM energy emitting devices are low level (class III) lasers, light emitting diodes (LEDs) and other semiconductor energy sources. For example, the emitting devices could be provided by LEDs formed from the flexible graphite material, which may be configured as a flexible form of LED. The emitted EM energy may be in therapeutically effective visible or invisible wavelengths. Each laser 18 has an emitting end 18E located at the insole's top surface 12T to direct laser energy onto the sole of a foot. In this example, each emitting end 112 may be flush with the insole's top surface 12T. The emitting end 18E may alternatively be spaced below the top surface 12T, with an air gap between the emitting end and the user's foot. In other examples, the laser emitting ends 18E might be covered by a top protective layer of material that is transparent to the emitted electromagnetic energy, to provide a protective barrier between the lasers and the foot.

Configuration features of the laser array 16 may be customized to a particular type of foot problem (e.g., heel ulcer, inner arch wound) or a foot problem of a particular patient. The configuration features may include type of lasers used, density of lasers (i.e., number of lasers per given area), location of the laser array along the insole top surface 12T (e.g., under the heel, under the inner arch, distributed over the insole's entire top surface). Configuration features might further include the shape of the array 16, such as an array in which the lasers are arranged in concentric circles or (as in this case) arranged in columns 21 and rows 22. Configuration features might also include spacing between adjacent columns 21 (column spacing), spacing between adjacent rows 22 (row spacing), and an angle 0 between the columns 21 and rows 22. In the example of FIG. 2, the angle 0 is 90 degrees in that the columns 21 are perpendicular to the rows 22, and column spacing equals row spacing. The array 16 may be positioned for the emitting ends 18E to be located at “trigger points” (i.e., treatment locations that have proven effective) for the relief of pain, increase in circulation, killing of bacteria or fungus, etc.

An internal laser activation unit 24, within the shoe 14, provides electricity to activate the lasers 18. In this example, the activation unit 24 is secured to the insole 12 at the insole's heel section which supports the user's heel. The activation unit 24 (as shown in FIG. 4) is embedded within the insole 12, in that it is spaced from both the top and bottom surfaces 12T, 12B, to provide an upper cushioning layer between the activation unit 24 and the top surface 12T and a lower cushioning layer between the activation unit and the bottom surface 12B.

The activation unit 24 includes an internal power supply 26 that supplies electrical power to the lasers 18. The internal power supply 26 may include an electrical charge storage device 27, such as a capacitor or rechargeable battery. The power supply may be recharged by electrical recharging current 28 from an external electrical source 30 (external to the shoe 14). Examples of the external electrical source are a cord plugged into a wall socket, a power adapter (e.g., 120VAC to 12VAC) plugged into a wall socket, a USB port (e.g., of a computer), a solar cell attached to an outside surface of a garment (such as the shoe) that converts ambient light to electricity, and a piezoelectric device (piezo strip) attached to a garment that converts body motion to electricity.

An electrical recharging link 32 extends from the external power source 30 to the internal power supply 26 to deliver the charging current 28. The recharging link 32 may be a cable. As shown in FIG. 3, the charging link 32 may alternatively include an EM induction mechanism, that includes a pickup coil 34, in the shoe 14, that is exposed to an alternating magnetic field from an electromagnet 35 powered by the external power source 30 outside the shoe wall 14.

The internal power supply 24 may include a power source internal to the shoe 14, so as to require less power or no power from the external power source 30. An example internal power source is a force transducer. The force transducer may be a piezoelectric device 36 (e.g., piezo strip) shown in FIG. 4. The piezo strip is located in the shoe 14 and lies flat and parallel with the insole's top and bottom surfaces 12T, 12B. It converts compression and bending of the insole 12 to electricity that adds to the stored charge in the charge storage device 27 and powers the lasers 18. The internal power supply 26 may then be charged by (and the lasers 18 powered by) each pace (step) of the user. The electricity for powering the laser treatment would then be generated only when the user is walking In that case, an on/off switch might not be necessary, since electricity would not be wasted on powering the lasers 18 when the insole 12 is not worn. The internal power supply 24 might accumulate power it draws from the piezo strip 36 for only sufficient duration to reach a threshold electrical charge sufficient to power a laser pulse. The time duration for achieving the threshold charge depends on how much charge (in volts, current or power) each of the user's steps adds to the stored charge and how much of the charge is withdrawn to produce a pulse. Depending on the duration for achieving the threshold charge, each step might power one pulse or multiple pulses, or multiple steps might be required to produce one pulse. In any of these cases, the charging duration might be less than a few seconds. For such short charging durations, a battery might be unnecessary, and a capacitor would be well suited. A capacitor, unlike a rechargeable battery, never degrades or needs replacing.

The piezo strip 36 may be powered by the activation unit 24 to vibrate during the laser treatment. This might enhance blood circulation during the treatment and also provide a tactile indication (notification) to the user that the EM energy is being applied. In this case, the piezo strip 36 charges the battery 27 from the foot's relatively slow moving pressure application (“slow” in that it occurs at the speed of taking a step) and later uses the charge to vibrate the foot tissue at relatively fast frequency of 5 Hz or greater.

Another example internal power source is a magnet-and-coil device 38 shown in FIG. 5. It includes a magnet 38M that is movable within a nonmagnetic tube 38T embedded in the insole 12 within the shoe 14. It further includes a pickup coil 38C surrounding the tube 38T that feeds the power supply 26. The magnet oscillates (moves back and forth) within the tube with each step of the user, due to oscillatory motion of the insole 12. Each oscillatory movement of the magnet 38M through the coil 38C generates an electrical pulse that charges the power supply 26 or directly powers the lasers.

A controller 40, of the activation unit 24, includes an electrical circuit that controls conduction of power from the power supply 26 to the lasers 18. The controller 40 in this example includes a microprocessor 41 that executes software code instructions that are stored in a memory device 42 (data storage device), to perform functions of the controller. The memory device 42 may also store data (such as from sensors) that is collected by the controller 40 during treatment sessions.

The controller 40 controls laser activation parameters. The activation parameters might include a starting time of a laser treatment and the treatment's duration (e.g., in seconds). If the energy is emitted in pulses, the activation parameters may include pulse duration and pulse rate (e.g., number of pulses per unit time). The activation parameters may also include laser output power, which is controlled by controlling power (via wattage, voltage or current) supplied to each laser 18.

The controller 40 may power different lasers 18 at different locations of the foot with different activation parameters.

The controller 40 may power the lasers 18 to pulse simultaneously, or alternatively in a staggered but coordinated manner. For example, the controller 40 may activate lasers 18 in a sequence, for the light pulses to be emitted in a moving wave of light (corresponding to a string of activated lasers) that travels along the insole surface 12T. The moving wave may provide a massaging effect that promotes blood flow. The wave can travel longitudinally, by pulsing a first row 22 of lasers 18, then the next row, and then the next row, etc. The wave can travel laterally, by pulsing a first column 21 of lasers, then the next column, and then the next column, etc. The wave can comprise a ring of light that travels radially outward from a starting (center) point, by first pulsing a first ring of lasers centered on a starting point, then pulsing a second ring of lasers immediately surrounding the first laser, and then pulsing a third ring of lasers immediately surrounding first ring, etc. The wave may comprise a ring of light that travels radially inward from an outer ring of lasers toward a center point, by first pulsing the outer ring of lasers, then pulsing a second ring of lasers located within the first ring, and then pulsing a third ring located within the second ring. In the radially outward and radially inward examples, each successively activated ring may be concentric or non-concentric with the previously activated ring. A pulse may travel (orbit) along a circle or oval, by pulsing each laser of a ring of lasers in sequential order about the ring. The radius of the ring can gradually increase to produce an outward spiral, or gradually decrease to produce an inward spiral.

The controller 40 may include a temperature sensor 43 that measures (senses) temperature of the foot surface and outputs a temperature signal indicative of the temperature. The controller 40 may control the laser treatment to start when the foot surfaces is below a threshold temperature and to end when the foot surface is above a threshold temperature.

The controller 40 may include a compression sensor 44 (force sensor) that measures (senses) compression force applied by the foot to the insole and outputs a data signal indicative of the compression force. The controller 40 may use the compression (force) data to count and record the number of paces (steps) the patient has walked. The controller 40 may also use the compression data to control the treatment to occur only when the foot compresses (presses against) the insole 12. This avoids the treatment device 10 wasting electricity to power the lasers 18 when a foot is not even in the shoe 14. And even when a foot is in the shoe 14 and the patient is walking, this compression-dependent feature limits generation of the laser energy to when it will penetrate deepest into the foot tissue and be most therapeutically productive, and avoids electricity usage and heat generation at times when the laser energy might be less productive. With this compression-dependent feature, the insole 12 might not require an on/off switch. The piezo strip 36 described above for powering the lasers 18 may serve as the compression sensor 44.

An external controller 50, outside the shoe 14, may send control data, such as laser activation parameters (e.g., start time, pulse duration) to the internal controller 40. The external controller 50 may be a personal computer with a software application configured to enable a medical practitioner (user of the external controller) to select laser activation parameters to be communicated to the internal controller 40. The external controller 50 may also receive feedback data from the internal controller 40. The feedback data may include battery charge, battery capacity, historic usage information such as when the treatments occurred, their durations, sensed foot temperature (from temperature sensor), and number of steps walked (from force sensor).

A data communication link 51 conducts communications between the external controller 50 and the internal controller 40. The communication link 51 may be a wired data communication line, such as the recharging electrical line 32 extending from the insole 12 to outside the shoe 14. Alternatively, the data communication link 51 may be a short range wireless communication link, such as Bluetooth.

An electrical supply line 52 conducts electricity (electrical power) from the activation unit 24, under control of the controller 40, to the lasers 18. In this example, the supply line 52 is a trace pattern (FIG. 2) of flexible conductive material. Examples of the flexible conductive material are (1) flexible graphite material, such as Papyex® Flexible Graphite sold by Mersen USA BN Corp. of Bay City, Mich., (2) carbon impregnated rubber and polymers, (3) gold nanoparticles that are embedded in a flexible synthetic polyurethane material, (4) semiconducting nanowires, (5) highly electrically conductive and highly flexible elastomer such as Metal Rubber™, (6) flexible electrically conductive non-wovens, (7) electrically conductive fabrics such as from KREMPEL, (8) material produced by impregnating non-woven, (9) fabric materials with bonding agents containing electrically conductive particles, (10) carbon nanotube-coated silicone, (11) carbon nanotube films, (12) graphene sheets and (13) metal-nanowire meshes.

The trace pattern 52 in this example is patterned from a sheet of flexible graphite material and included as an inner layer within the shoe insole 12. The flexible graphite trace layer 52 may dissipate heat generated from the lasers 18 to protect the user's foot from discomfort or thermal damage. The supply line 52 is sandwiched between upper and lower insulating layers 54U, 54L.

A heat expelling component 53 may conduct heat from inside the show 14 to outside the shoe 14. The heat expelling component 53 is of heat conducting material, such as a metal strip or graphite material. The heat expelling material 53 may be an extension of the graphite material that forms the electrical supply line 52 to the lasers 18, and may extend from the supply line 52 inside the shoe 14 to a location outside of the shoe 14. If the footwear is a sandal or sock, which enables ventilating air to reach the foot, then a heat expelling material may be less beneficial than if the footwear is a shoe.

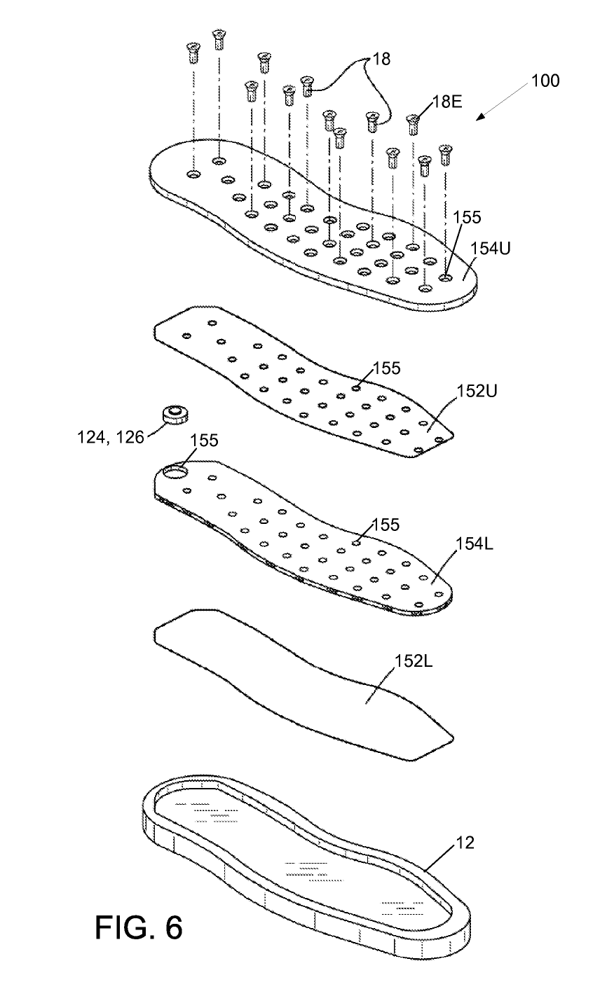

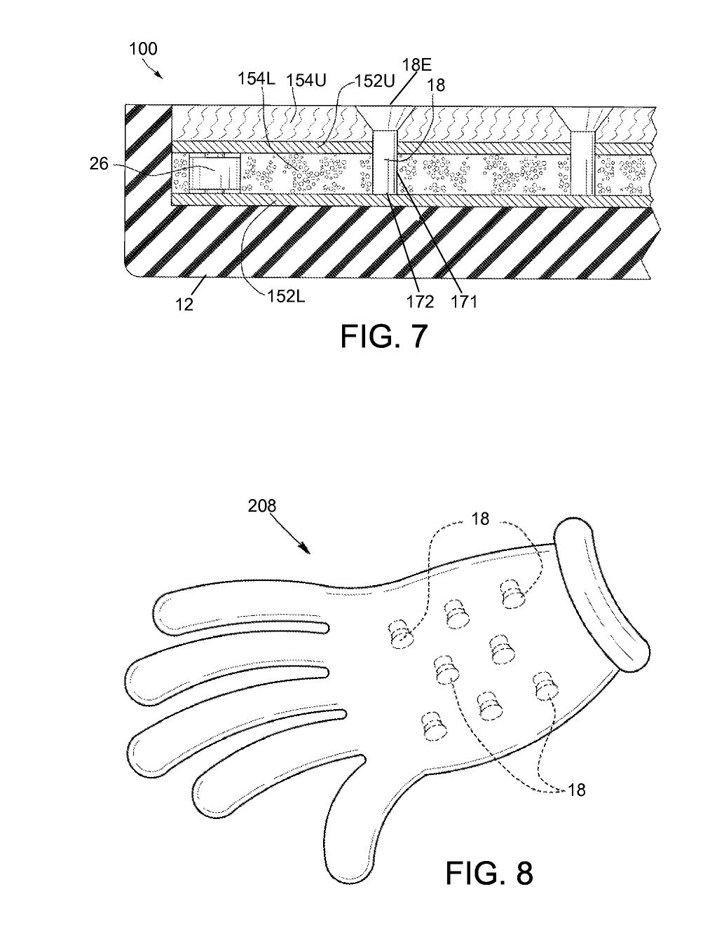

FIGS. 6 and 7 are respectively an exploded view and an assembled view of a second example foot treatment device 110 for use in footwear (e.g., shoe, sandal, sock). This second treatment device 110 is similar to the first foot treatment device 10 of FIGS. 1-5. The second treatment device 110 includes the array 16 of therapeutic lasers 18 like that of the first treatment device 10. The second device 110 differs from the first device 10 in that its activation unit 124 does not include a controller, but includes only the power supply 126 which in this example comprises a button battery. The second device 110 differs further from the first device 10 in that the supply line that conducts electricity from the activation unit 124 to the lasers 18 comprises upper and lower layers 152U, 152L of flexible electrically conductive material within the insole 112. The supply line layers 152U, 152L may include, for example, any of the thirteen examples listed above for the first device, and in this example is flexible graphite material. One of the upper layer and a lower layers 152U, 152L conducts electricity from the activation unit 24 to the lasers and the other conducts the electricity from lasers back to the activation unit 24. Each of the upper and lower layers 152U, 152L has a longitudinally extending length and a laterally extending width that define an area within which all of the lasers 18 are located. Accordingly, the periphery of each layer 152U, 152L encompasses all of the lasers 18.

In this example, the upper layer 152U provides a power plane for the lasers 18, and the lower layer 152L provides a ground plane. The lasers 18 are inset through holes 155 in the upper and lower insulating layers 154U, 154L and upper supply line layer 152U, and contact the upper and lower conductive layers 152U, 152L. The battery 126 is tightly held in a hole 155 in the lower insulating layer 154L, and its terminals contract the upper and lower supply line layers 152U, 152L. As with the supply line 52 of the first treatment device 10, the supply line layers 152U, 152L of the second device 110 may dissipate heat generated from the lasers 18 to protect the patient's foot from discomfort or thermal damage. Further protection from heat emitted by the lasers 18 may be provided by the insulating layers 154A, 154B.

Each laser 18 includes power and ground terminals 171, 172 that are respectively positioned to provide contact with the supply line layers 152U, 152L. In this example, the laser's power terminal 171 is located at the side of the laser 18 to connect to the power plane 152U, and the ground terminal 172 is located at the bottom of the laser 210 to connect to the ground plane 152L.

FIGS. 8-11 illustrate other example mechanisms for delivering therapeutic EM energy to a patient utilizing therapeutic lasers capable of emitting EM energy that may provide, for example, biostimulation and/or a reduction of pain, bacteria, or onychomycosis.

In FIG. 8, therapeutic lasers 18 are located in a glove 208 (glove insert) for treating a hand with EM energy.

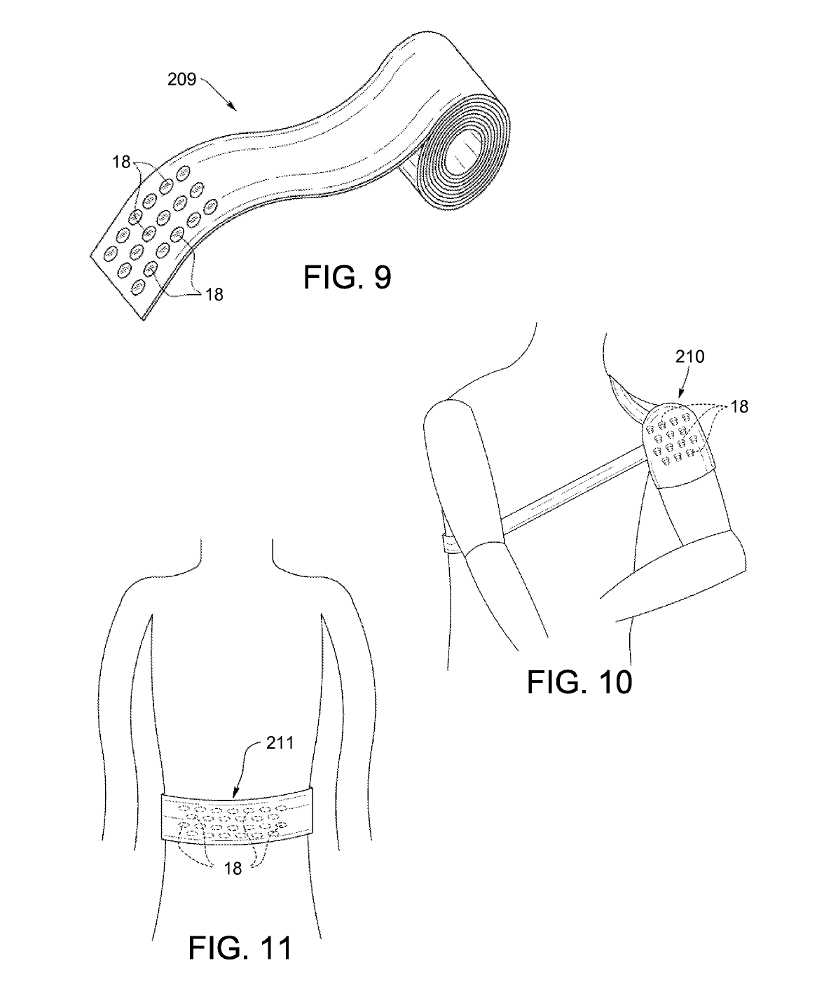

In FIG. 9, therapeutic lasers 18 are located in a bandage 209 for treating a body part about which the bandage is wrapped with EM energy.

In FIG. 10, therapeutic lasers 18 are located in a compression sleeve 210 worn on a patient's shoulder for treating the shoulder with EM energy.

In FIG. 11, therapeutic lasers 18 are included within a garment 211 that positions the lasers 18 adjacent the patient's lower back for treating the back with EM energy.

In the examples of FIGS. 8-11, the lasers 18 may be arranged such that, when positioned against the patient's body, the lasers' emitting ends (18E in FIG. 4) are located at “trigger points” (i.e., treatment locations that have proven effective) for the relief of pain, increase in circulation, killing of bacteria or fungus, etc. Other examples might include garments or other mechanisms for positioning therapeutic electromagnetic energy emitting devices at other parts of the body that may benefit from, for example, biostimulation of the tissue, blood flow and/or blood vessels. Examples of other parts of the body that may benefit include hemorrhoids, the prostate, and other male and female genitalia, and their related medical indications.

Similar to the examples of FIGS. 1-7, the lasers 18 of FIGS. 8-11 may be coupled to their power source (126 in FIG. 1) by a flexible graphite material (not shown). Also, like the examples of FIGS. 1-7, the lasers 18 in FIGS. 8-11 are preferably high level (class IV) therapeutic lasers, but other sources of therapeutic electromagnetic energy could also be used, such as low level (class III) lasers, LEDs or other semiconductor energy sources. For example, therapeutic electromagnetic energy could be provided by LEDs formed from the flexible graphite material (which may be configured as a flexible form of LED.) In addition, the lasers 18 may be in the visible or invisible spectrum, and exhibit efficacious properties.

The example laser treatment devices described above enable therapeutic advantages of a high level laser to be implemented in a treatment schedule over an extended period, for example to accelerate healing of an injury, to provide pain relief, and/or to kill fungus or bacteria. Despite the high power requirements of a high level laser, the configurations described above reduce the bulkiness that would otherwise be required.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents. For example, other embodiments could include therapeutic lasers and a flexible graphite interface within other types of garments, such as a compression arm or leg sleeve, a vest, a sock, an adhesive patch, etc. In another example, a high power (class IV) laser source could be utilized with a plurality of optical fibers in a bundle that terminate into optical lens that can be selected so the electromagnetic energy pattern penetrates deeply (low divergence angle) for certain conditions such as acute shoulder injuries and spreads widely for other conditions like nail fungus or acne.

The components and procedures described above provide examples of elements recited in the claims. They also provide examples of how a person of ordinary skill in the art can make and use the claimed invention. They are described here to provide enablement and best mode without imposing limitations that are not recited in the claims. In some instances in the above description, a term is followed by a substantially equivalent term enclosed in parentheses.

1. A therapeutic device comprising:
a garment configured to be worn on a user's body over a treatment area;
a plurality of therapeutic electromagnetic (EM) energy emitting devices, fixed to the garment at locations within the garment for irradiating the treatment area with EM energy when the garment is worn over the treatment area;
a power supply, fixed to and within the garment, that stores electrical charge and outputs electrical supply current from the stored electrical charge; and
a flexible graphite material that is located within the garment and that conducts the electrical supply current to the EM energy emitting devices to activate the EM energy emitting devices.

2. The therapeutic device of claim 1, wherein the EM energy emitting devices are high level class IV lasers.

3. The therapeutic device of claim 1, wherein the EM energy emitting devices are low level class III lasers.

4. The therapeutic device of claim 1, wherein the EM energy emitting devices are light emitting diodes (LEDs).

5. The therapeutic device of claim 1, wherein the garment is a shoe insole.

6. The therapeutic device of claim 5, wherein the device further includes a controller configured to control the EM emitting devices to be activated in a sequence.

7. The therapeutic device of claim 6, wherein the sequence is in a longitudinal direction of the insole or radial direction of the insole.

8. The therapeutic device of claim 6, wherein the sequence yields a ring of light that travels radially outward from a center point or radially inward toward a center point.

9. The therapeutic device of claim 6, wherein the power supply includes a piezoelectric device that is located in the insole and that generates, from compression and bending of the insole, electrical charge current for charging the power supply.

10. The therapeutic device of claim 9, further comprising a controller configured to control the electrical supply current to power the piezoelectric device so as to cause the piezoelectric device to vibrate when the EM energy emitting devices are activated.

11. The therapeutic device of claim 6, wherein the power supply includes a movement transducer that is located in the insole and that generates, from oscillatory movement of the insole, electrical charge current for charging the power supply.

12. The therapeutic device of claim 6, further comprising:
a temperature sensor, in the insole, configured to sense temperature; and
a controller, in the insole, configured to control activation of the EM emitting devices based on the sensed temperature.

13. The therapeutic device of claim 6, further comprising:
a compression sensor, in the insole, configured to sense compression force applied by a foot to the insole; and
a controller, in the insole, configured to control activation of the EM emitting devices based on the sensed compression and to count a number of steps taken with the shoe insole based on the sensed compression.

14. The therapeutic device of claim 6, further comprising:
a shoe in which the insole is located;
an internal controller, in the insole, configured to control activation of the EM emitting devices; and
an external controller, located outside the shoe, configured for enabling a user of the external controller to select treatment control parameters to be communicated to the internal controller through a wireless communication link between the internal controller and the external controller.

15. The therapeutic device of claim 6, wherein
the flexible graphite material includes an upper layer and a lower layer, one of which configured to conduct the electrical supply current to the EM energy emitting devices and the other of which configured to conduct electrical current from the EM energy emitting devices;
each of the upper and lower layers has a length and width that define an area within which the plurality of the EM energy emitting devices are located; and
each of the EM energy emitting devices is contained in a hole in the upper layer and has a side that electrically contacts the upper layer and has a bottom that conducts the lower layer.

16. The therapeutic device of claim 1, wherein the garment is a glove insert.

17. The therapeutic device of claim 1, wherein the garment is a bandage.

18. The therapeutic device of claim 1, wherein the garment is configured to be worn a shoulder.

19. The therapeutic device of claim 1, wherein the garment is configured to be worn a lower back.

20. A therapeutic device comprising:
a garment configured to be worn on a user's body over a treatment area;
a plurality of therapeutic electromagnetic (EM) energy emitting devices, fixed to the garment at locations within the garment for irradiating the treatment area with EM energy when the garment is worn over the treatment area;
a power supply, fixed to and within the garment, that stores electrical charge and outputs electrical supply current from the stored electrical charge; and
a flexible conductive material that is located within the garment and that conducts the electrical supply current to the EM energy emitting devices to activate the EM energy emitting devices;
wherein the flexible conductive material comprises at least one of carbon impregnated rubber, carbon impregnated polymer, gold nanoparticles embedded in polyurethane, semiconducting nanowires, a non-woven, an electrically conductive fabric, carbon nanotube-coated silicone, carbon nanotube film, graphene sheets, and metal-nanowire mesh.
